(19) United States
(12) Patent Application Publication
(10) Pub. No.: US 2005/0020898 A1
(43) Pub. Date: Jan. 27, 2005

(54) System and method for configuring a scanning procedure

(76) Inventors: Kenneth J. Vosniak, Milwaukee, WI (US); Sridhar S. Madhavan, Waukesha, WI (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/616,353

(22) Filed: Jul. 10, 2003

(51) Int. Cl. A61B 5/05

(52) U.S. Cl. 600/407

(57) ABSTRACT

According to one embodiment, the invention relates to a method of configuring a scan in an imaging device comprising the steps of beginning a data acquisition step for a first scan, during the data acquisition step for the first scan, completing a data entry step relating to a second scan, completing the data acquisition step for the first scan, and beginning a data acquisition step for the second scan. The invention also relates to an imaging system comprising a detector which detects radiation during a data acquisition step of a scan, at least one processor which controls configuration and execution of the scan, and at least one memory which stores at least one computer program for executing the scan and data for configuration of the scan, wherein the processor is programmed to conduct the data acquisition step for a first scan, and during the data acquisition step for the first scan, conduct a data entry step for a second scan.

### BACKGROUND

The present invention relates generally to imaging, and more particularly to a system and method for configuration of a scanning procedure for an imaging device.

Hospitals and other health care providers rely extensively on imaging devices such as CT scanners, MRI scanners and PET scanners for diagnostic purposes. These imaging devices have been improved over the years to provide high quality images of various bodily functions and structures. Of course, due to their complexity, such imaging devices are quite expensive. It is beneficial, therefore, for a hospital or other purchaser of such equipment, to use it efficiently so as to recoup its investment.

Before performing a scan, certain information on the scan and the patient must be entered into the scanner memory to control the scan. In many hospitals, some of this information is stored in a central database sometimes referred to as a Hospital Information System (HIS). Certain data from the HIS can be downloaded to the memory of the scanner. However, it is also necessary for an operator of the scanner to enter additional information prior to the scan. For example, the scanner operator may need to enter patient-specific information about the scan, such as tracer injection information in a PET scan. The operator may also need to specify one or more modality specific scan protocols to define the parameters of the scanning procedure. The operator typically enters this information manually into the scanner after the patient arrives at the scanner for the scanning procedure. The operator may also need to view a list of patients in the HIS to determine and select the next patient to be scanned.

During the time that the operator enters data into the scanner with a keyboard before a scan, or examines a patient list in the HIS to select the patient to be scanned, the scanner is not acquiring data, which reduces its operating efficiency. Depending on how much information must be manually entered by the operator, the efficiency with which the scanner is used to acquire medical imaging data may be significantly decreased. For example, the data entry process before initiation of a scan may take 25%-40% of the time that the scanner is otherwise available for scanning. The present invention addresses this and other drawbacks of known systems.

### SUMMARY

According to one embodiment, the invention relates to a method of configuring a scan in an imaging device comprising the steps of beginning a data acquisition step for a first scan, during the data acquisition step for the first scan, completing a data entry step relating to a second scan, completing the data acquisition step for the first scan, and beginning a data acquisition step for the second scan. The invention also relates to an imaging system comprising a detector which detects radiation during a data acquisition step of a scan, at least one processor which controls configuration and execution of the scan, and at least one memory which stores at least one computer program for executing the scan and data for configuration of the scan, wherein the processor is programmed to conduct the data acquisition step for a first scan, and during the data acquisition step for the first scan, conduct a data entry step for a second scan.

According to another embodiment, the invention relates to a method for configuring an imaging device comprising the steps of specifying at least one criterion for determining a next patient to be scanned from a plurality of scheduled patients, querying a database with the at least one criterion, and receiving an identification of the next patient to be scanned based on the at least one criterion. The invention also relates to an imaging system comprising a detector which detects radiation during a data acquisition step of a scan, at least one processor which controls configuration and execution of the scan, and at least one memory which stores at least one computer program for executing the scan and data for configuration of the scan, wherein the processor is programmed to allow an operator to specify at least one criterion for determining a next patient to be scanned from a plurality of scheduled patients, query a patient database with the at least one criterion, and receive an identification of the next patient to be scanned based on the at least one criterion.

The invention also relates to an article of manufacture which comprises a computer usable medium having computer readable program code means embodied therein for causing a computer to execute the methods described herein relating to configuring a scan.

### BRIEF DESCRIPTION OF THE DRAWINGS

FIG. 1 is a drawing of an imaging system according an exemplary embodiment of the invention;

FIG. 2 is a schematic diagram of data and software stored in the memory 36 of FIG. 1 according to an exemplary embodiment of the invention;

FIG. 3 is a screen display of a list of patients scheduled for scanning procedures according to an exemplary embodiment of the invention;

FIG. 4 is a flow chart which illustrates a method of configuring a scan according to an exemplary embodiment of the invention;

FIG. 5 is an example of a screen showing scan data during the data acquisition phase of a scan according to an exemplary embodiment of the invention;

FIG. 6 is an example of a screen which allows an operator to enter tracer information for a patient according to an exemplary embodiment of the invention;

FIG. 7 is an example of a screen which allows an operator to enter scan protocol information according to an exemplary embodiment of the invention;

FIG. 8 is an example of a screen which allows an operator to view and edit patient information according to an exemplary embodiment of the invention;

FIG. 9 is an example of a screen which allows an operator to add a patient to the schedule according to an exemplary embodiment of the invention;

FIG. 10 is a diagram showing a preferences screen and database which can be used to specify scan order preferences for patients according to an exemplary embodiment of the invention; and

FIG. 11 is a diagram showing a method of sending a next patient query to a database according to an exemplary embodiment of the invention.

### DETAILED DESCRIPTION

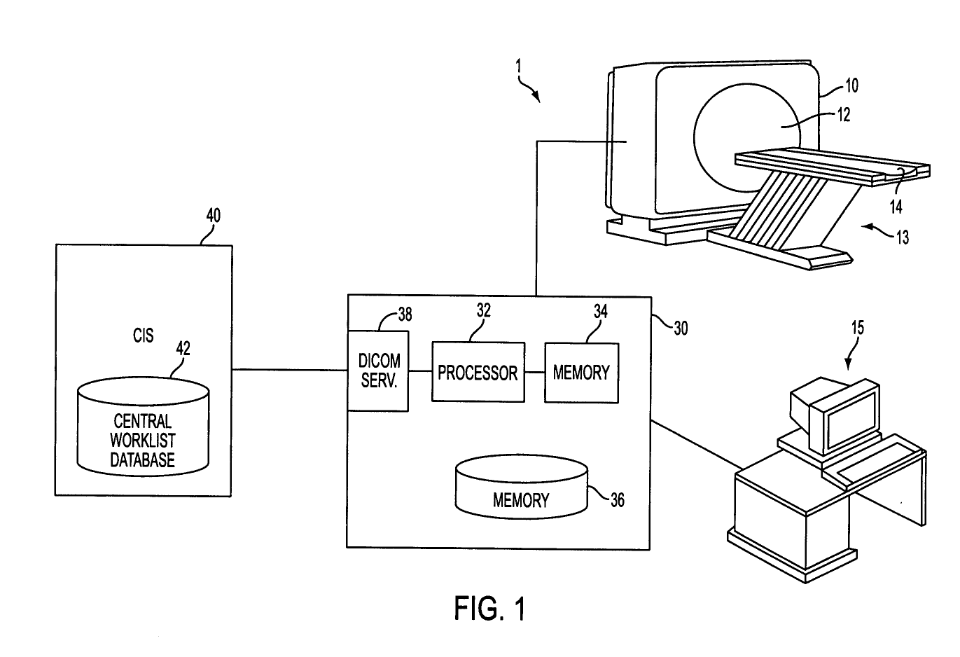

FIG. 1 illustrates an example of a scanner 1 which includes a gantry 10 supporting a detector about a central opening or bore 12. The scanner 1 may be a positron emission tomography (PET) scanner, a single photon emission computed tomography (SPECT) scanner, a computed tomography (CT) scanner, a magnetic resonance imaging (MRI) scanner, for example, or another imaging device such as an X-ray imager. A patient table 13 is positioned in front of the gantry 10. A patient table controller (not shown) moves the table bed 14 into the bore 12 in response to commands received from an operator workstation 15. The operator workstation 15 includes a display and a keyboard. Through the keyboard and associated control panel switches, the operator can control the operation of the scanner 1 and the display of the resulting image on the display.

FIG. 1 also shows a processing unit 30 which includes a processor 32, a first memory 34, and a second memory 36. The first memory 34 may comprise random access memory (RAM), for example, which stores programs being executed by the processor 32. The second memory 36 may comprise a mass memory such as a hard drive or other mass storage device, which stores data and programs. The processing unit 30 controls the operation of the scanner, including the particular scan protocol being executed, and stores the data acquired during the scan.

The processing unit 30 is connected to a central information system (CIS) 40 which may store patient demographic data, scheduling data, scan procedure data, patient medical history, visit histories, admission and discharge information, referrals, orders, results, prescription information, and/or diet information, for example. Some or all of this data can be stored in a central worklist database 42, as shown in FIG. 1. The CIS may comprise a Hospital Information System (HIS), a Radiology Information System (RIS), or other depository or database of information which stores and transmits patient information.

The processing unit 30 may also include a DICOM server 38. DICOM (Digital Imaging and Communications in Medicine) is a standard interface for connecting medical imaging equipment. The DICOM standard specifies the network protocol by which two DICOM-compatible systems communicate. The DICOM standard covers a broad range of medical imaging applications, for instance the transfer of images generated by a scan, transfer of reports generated from scan processing, or the transfer of worklist information from a scheduling system to a scanner. The specific information sent by the CIS 40 and the network protocol used to send this data are defined by the DICOM standard according to an exemplary embodiment of the invention.

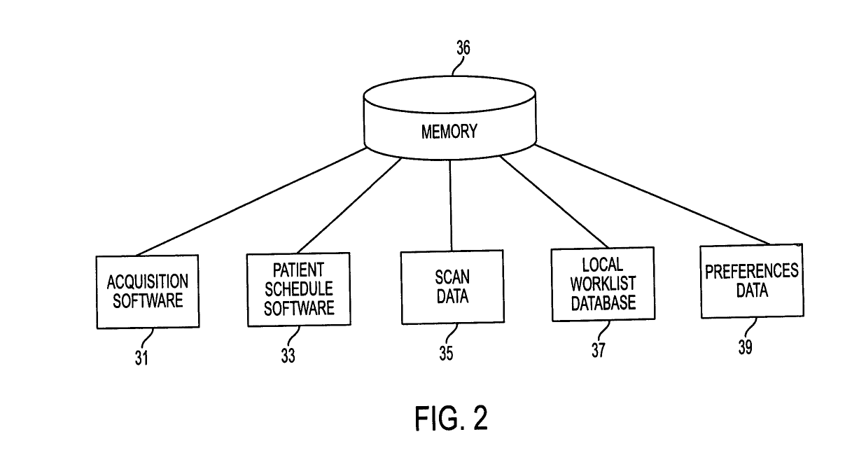

Referring to FIG. 2, the memory 36 in the processing unit 30 stores a number of software programs or subroutines which control the operation of the scanner 1. For example, the memory 36 stores acquisition software 31 for controlling the data acquisition process during a scan according to a scan protocol. The memory 36 also stores patient schedule software 33 which determines a next scheduled patient according to one or more criteria specified by the operator of the scanner 1. The patient schedule software 33 and the acquisition software 31 are typically integrated so that they run simultaneously during operation of the scanner 1.

The memory 36 also stores a number of types of data. For example, the memory 36 stores scan data 35 obtained from the data acquisition phase of the scan. As one example, the memory 36 may store a histogram which contains the scan data resulting from a PET scan which is used to reconstruct an image of the patient.

The memory 36 also stores a local worklist database 37. The local worklist database 37 stores the worklist information received from the CIS 40, as well as information entered locally by the operator of the scanner 1. The worklist information may include patient demographic information and other information pertaining to the patient (e.g., patient allergies, pregnancy status, etc.) and to the exam (e.g., exam description, referring physician, etc.).

The local worklist database 37 may also include the specific scan protocol(s) to be used during the scan (e.g., the specific instructions and parameters used to control the scan and the image reconstruction), as well as radioactive tracer information. The tracer information may include, for example, pre-injection assay information such as tracer activity (e.g., specified in units of milli-curies (mCi) and Mega-Becquerels (MBq)), the date and time that the tracer activity was assayed, the tracer volume, and a batch description, the time that the tracer was injected into the patient, and post-injection assay information such as tracer activity and the time of the assay. The tracer information typically must be entered locally (rather than being obtained from the CIS 40) because this information is not generally known when the patient worklist data is originally entered into the CIS, but rather is determined during pre-scan procedures.

According to exemplary embodiments of the invention, the patient scheduler software 33 runs independently of and concurrently with the acquisition software 31, which allows the operator of the scanner to enter the tracer injection information for one patient while the acquisition software 31 scans another patient. Similarly, since the protocol information is often not known or entered at the time of patient scheduling, exemplary embodiments of the invention allow protocol information to be locally entered while the scanner is scanning another patient.

The memory 36 also stores user preferences and criteria in a preferences database 39 for determining a patient scanning order, as will be described further below.

Of course, the arrangement shown in FIGS. 1 and 2 is merely an example. The scanner 1, for example, may include more than one processor or memory to perform the various functions, and these components may be located at other locations such as the gantry 10, the work station 15, or at another server or processing unit. The system can be configured as desired, as will be appreciated by those skilled in the art.

A method of configuring a scanning procedure will now be described according to an exemplary embodiment of the invention.

Initially, the operator of the scanner 1 typically submits a query to the CIS 40 to obtain a list of patients to be scanned. For example, the operator may send a query to the CIS 40 at the beginning of each day, to obtain a list of patients to be scanned at that scanner on that day. The CIS 40, which typically contains a scheduled exam time for each patient in the hospital, sends the requested list to the processing unit 30 of the scanner 1. The CIS 40, in addition to sending the names of the patients, typically sends additional information to populate the local patient worklists in the local worklist database 37 for the relevant patients. For example, the CIS 40 can send one or more of: birth date, gender, height, weight, occupation, pregnancy status and other demographic data, patient identification number, scheduling data such as scheduled exam time, scan procedure, patient medical history, visit histories, admission and discharge information, referrals, physician name, orders, results, prescription information, symptoms, and/or diet information, for example. In the context of obtaining worklist information from a CIS, information which can be sent by the CIS is described, for example, in the DICOM 3.0 Modality Worklist Information Model. The DICOM Modality Worklist Model can be found in DICOM Annex K: Basic Worklist Management Service (PS 3.4 2001). Other relevant sections of the DICOM Standard pertaining to Worklist information are DICOM Supplement 10 (Basic Worklist Management), DICOM Part 3 Addendum (Basic Worklist Management), DICOM Part 4 Addendum (Basic Worklist Management), and DICOM Part 6 Addendum (Basic Worklist Management).

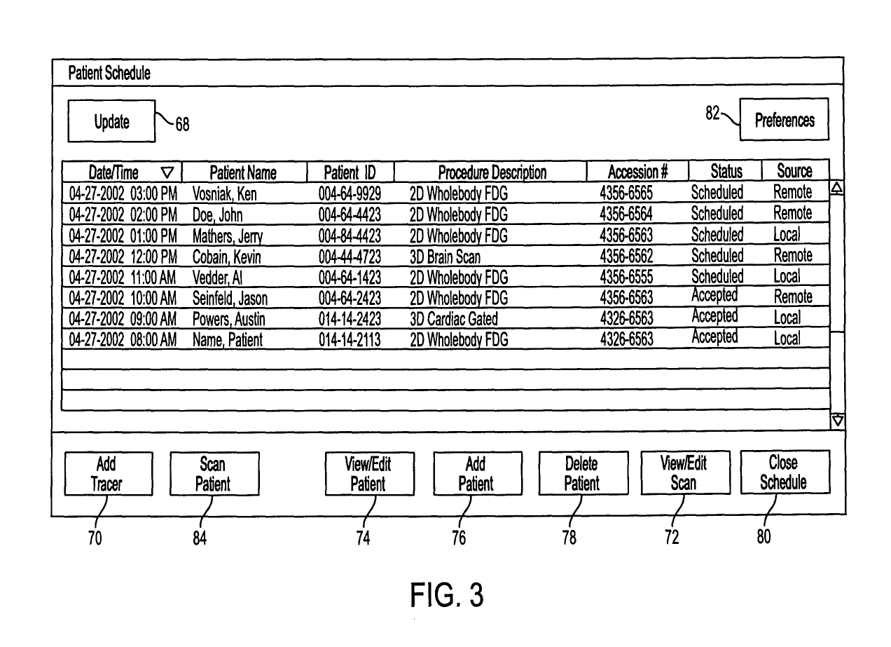

Once the list of patients and any associated worklist data are stored in the local worklist database 37, the operator can display the patient list on the display of the work station 15. FIG. 3 is an example of a screen showing a list of patients for the day for a particular scanner and the scheduled exam times.

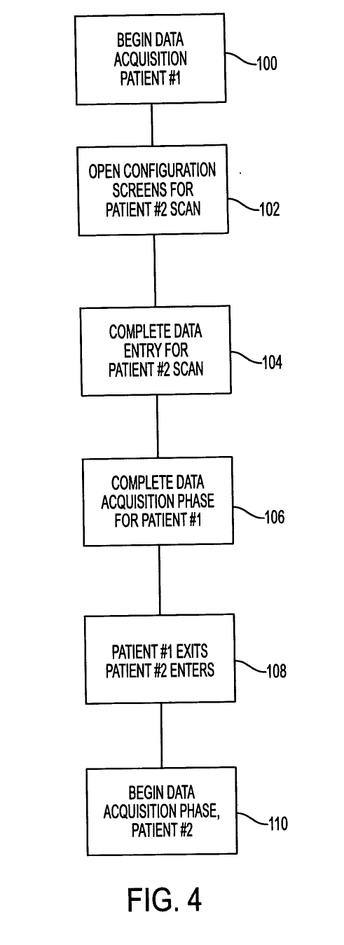

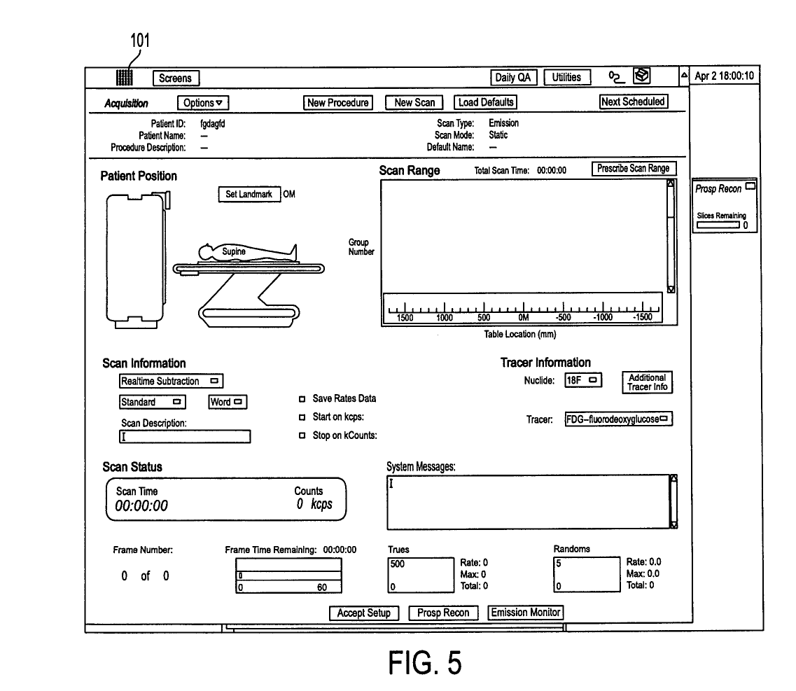

One feature of exemplary embodiments of the invention which can provide increased efficiency relates to data entry during the data acquisition phase of a scan. The data acquisition phase refers to the phase when the patient is undergoing the scan and the scanner is acquiring the scan data, e.g., the data to reconstruct an image of the patient. FIG. 4 is a flow chart illustrating an example of a method for configuring a scan during the data acquisition phase of an earlier scan. In step 100, the operator commands the scanner 1 to begin the data acquisition phase for a first scan which has previously been configured for a first patient. The operator can make this command, for example, by clicking button 84 in FIG. 3. In response to this command, the scanner 1, using the acquisition software 31, executes the scan according to the scan protocol, acquires the scan data, and stores the scan data in the database 35. During the scan, the acquisition software 31 can display various data on the progress of the ongoing data acquisition phase. For example, a screen such as shown in FIG. 5 displays scan information such as a scan description, scan status, frame number, time remaining, scan range, and tracer information, among other things.

During the data acquisition phase of the scan of the first patient, the operator, in step 102 (FIG. 4), clicks a button on the display of the work station 15 to configure a scan for a second patient. For example, as shown in FIG. 5, the operator can click button 101, which is visible and available during the data acquisition phase, to begin configuring another scan. The operator can thus prescribe ahead the scan configuration for subsequent patients during the data acquisition phase of an earlier patient. The button 101 may be labeled appropriately to indicate its function (although it is not labeled in FIG. 5). The button 101 may be programmed to display the list of patients shown in FIG. 3, for example, to allow the operator to select a desired patient from the list. Once the operator has selected a desired patient, the operator can view screens which display all of the information related to configuring the scan for that patient. For example, the operator can add tracer information such as injection time, time of assay, and amount of tracer activity, view or edit the patient data, view or edit the scan protocol data, or view or add any other data necessary to complete the configuration of the scan for the second patient or other subsequent patient.

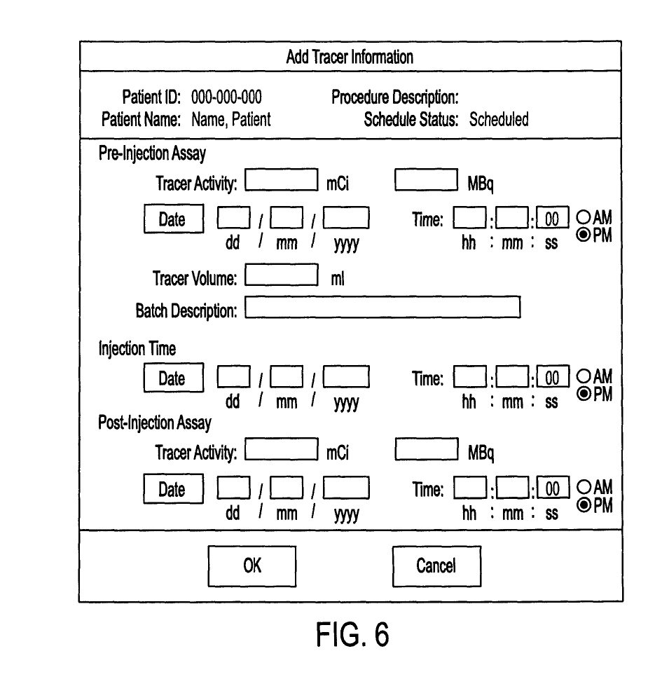

Referring to FIG. 3, the operator can add tracer information by clicking button 70. If the operator clicks button 70 to add or edit tracer information, the patient schedule software 33 causes a screen such as that shown in FIG. 6 to be displayed on the work station 15. The screen shown in FIG. 6 allows the operator to enter pre-injection assay information, including tracer activity, the date and time that the tracer activity was assayed, the tracer volume, and a batch description, the time that the tracer was injected into the patient, and post-injection assay information such as tracer activity and the time of the assay.

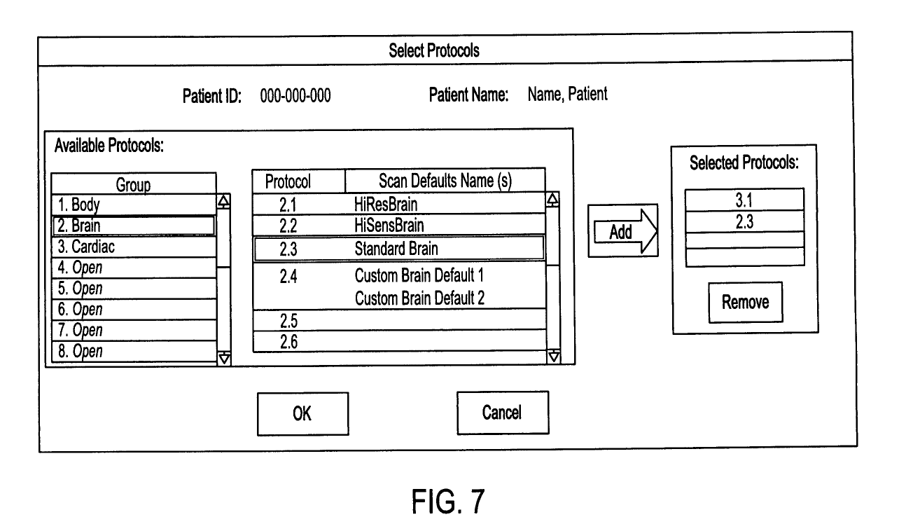

If the operator clicks button 72 in FIG. 3 to add or edit scan specific information, the patient schedule software 33 causes a screen such as that shown in FIG. 7 to be displayed on the work station 15. As shown in FIG. 7, the operator can select predefined scan protocols using a menu system. A scan protocol may comprise specific instructions that have been previously specified and saved as a template, for example. A particular scan protocol may specify parameters such as the length of the scan, the mode of the scan, etc., which may be applicable to some subset of patients being scanned. The scan protocols may be provided as part of the initial installation of the scanner, or generated by a physician or scanner operator based on their own desired settings. The particular protocol used depends on the type of scan requested and the purpose for the scan. A previously saved scan protocol can thus relieve the operator from entering the same scan parameters each time a patient requires that particular protocol. If the scan protocols have been previously generated and stored, the patient scheduling software 33 allows the scanner operator to selected the desired scan protocols at any time, for example, prior to the start of the scan, using the screen shown in FIG. 7, for example. The scheduler software 33 is decoupled from the acquisition software 31, which allows the operator to enter this information for any patient while another patient is being scanned, thus reducing set up time between patients.

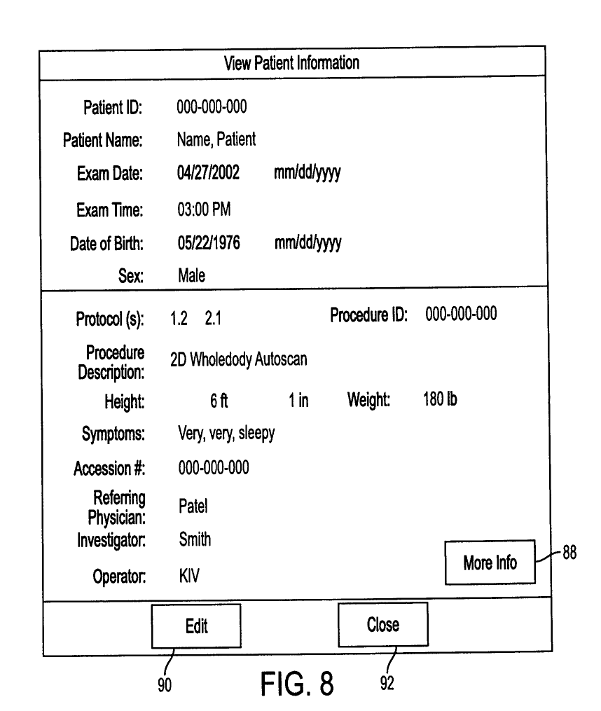

During the data acquisition phase, the operator can also perform other operations such as viewing or editing patient information (button 74), adding a new patient (button 76), deleting a patient (button 78), or closing the schedule (button 80). If the operator chooses to view or edit patient information, the patient schedule software causes a screen to displayed such as that shown in FIG. 8. The screen may display information such as patent ID, patient name, exam date and time, date of birth, sex, protocols, procedure ID, procedure description, height, weight, symptoms, accession number, referring physician, investigator, and operator. The patient information screen may also include a button 88 to allow the operator to see more information and a button 90 to allow the operator to edit the information. When the operator is finished viewing or editing the patient information, the operator clicks a button 92 to close the patient information screen.

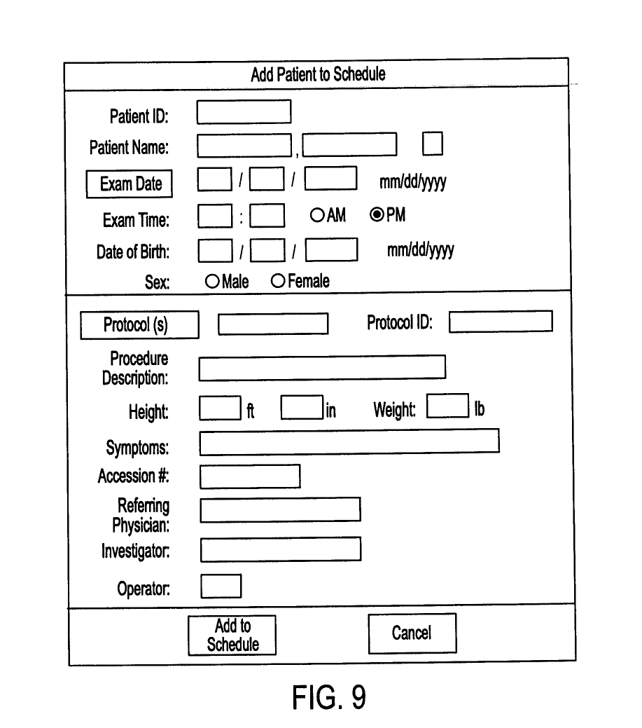

If the operator chooses to add a new patient, the patient schedule software 33 causes a screen to be displayed on the work station such as shown in FIG. 9. This screen may contain input boxes which allow the operator to enter all the information described above with respect to FIG. 8.

The operator can also submit a query to the CIS 40 at any time such as during the data acquisition phase, for example to obtain an updated list of patients for a particular scanner over a specified time period. The list of FIG. 3 can be updated at any time by the operator by clicking on an update button 68. One notable feature of the DICOM Standard Modality Worklist Information is that it only allows information known at the time of the patient scheduling to be automatically transferred to the scanner. The patient scheduling software 33, which decouples the DICOM Modality Worklist interface from the scanning software, allows the scan operator to enter scan-specific or patient-specific information while the scanner 1 is scanning other patients.

Referring again to FIG. 4, in step 104, the operator completes the data entry for the scan for the second patient. The data is stored in the local worklist database 37. As described above, the data entry may involve electronic transmission of data from the CIS 40 to the scanner 1 as well as additional data entry by the operator at the scanner. Typically, the operator can complete the data entry of step 104 for the second patient before the data acquisition phase has ended for the first patient. In step 106, the data acquisition phase for the first patient ends.

When the data acquisition phase of the first patient is completed in step 106, the scanner is ready to start the data acquisition phase of the second patient immediately. After the first patient leaves and the second patient is positioned in the scanner in step 108, the operator can initiate the second scan essentially immediately in step 110, because the entry of data for the second patient has already been completed by the operator.

To further enhance the efficiency of the scanner, the patient schedule software 33 may include a feature for automatically configuring the scanner (e.g., loading the previously entered data) for the second patient at the conclusion of the scan of the first patient. This feature allows the operator to specify one or more criteria for determining the next patient from the list of patients stored in the local worklist database 37. The processor 32 can then determine the next patient according to the stored criteria and automatically configure the scanner for that patient by retrieving the necessary patient information from the local worklist database 37.

As noted above, each patient in the list of patients to be scanned typically has an associated scheduled exam time which was previously entered in the CIS 40. The scheduled exam time, however, is not necessarily the best indication of the order in which the patients should be scanned. For example, if a patient fails to show up or arrives late, then it may be more efficient to scan a later scheduled patient first. Also, some scans require pre-scan procedures to be conducted. For example, in a PET scan, the patient is initially injected with a radioactive tracer, e.g., FDG. Therefore, the time of injection of the tracer may be a better indication of the order of patient scanning for a particular scanner than the scheduled exam time.

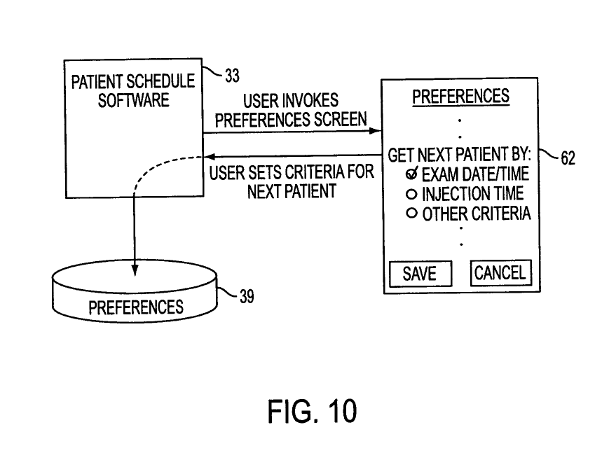

Referring to FIG. 10, the patient scheduling software 33 includes a functionality to cause a user preferences screen 62 to be displayed on the work station 15. The user preferences screen 62 allows the operator to specify one or more criteria for determining the next patient to be scanned. The criteria may include a tracer injection time, a scheduled exam time, the time of patient arrival, the time of patient registration, or a relative ordering determined by the scanner administrator, for example. The user specifies one or more criteria on the user preferences screen 62 and the user's selections are stored in the preferences database 39, which may form a portion of the memory 36 or a separate database.

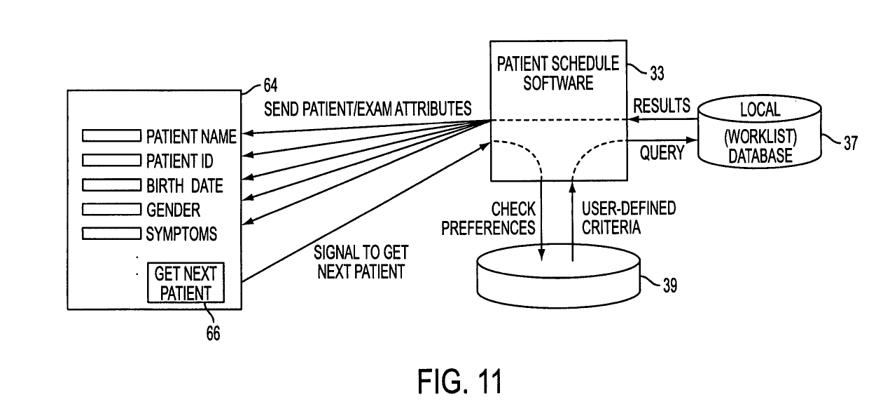

Referring to FIG. 11, the patient schedule software 33 applies the criteria to determine the next patient from the list of patients stored in the local worklist database 37. For example, at the start of each patient exam, the operator can click a button 66 on the work station screen to cause the patient schedule software 33 to determine the next patient based on the criteria stored in the preferences database 39. In this case, as shown in FIG. 11, the patient schedule software 33 retrieves the user defined preferences from the preferences database 39 and uses the preferences to construct a query directed to the local worklist database 37. In response to the query, the local worklist database 37 produces results in the form of identifying the next patient and providing worklist data associated with that patient. For example, as shown in FIG. 11, the patient schedule software 33 causes a patient screen to be displayed which, in addition to specifying the name of the patient, also shows a patient ID, birth date, gender, symptoms, and other desired data from the local worklist database 37 such as patient, exam, and tracer information.

The patient schedule software 33 can thus provide the advantage of automatically determining the next patient based on the operator's criteria. This functionality relieves the operator from manually retrieving a list of scheduled patients from the CIS and determining which patient should be the next patient, based on, for example, the scheduled exam time, the tracer injection time, the arrival time of the patient, and/or other factors. The functionality for identifying the next patient can also include the capability of automatically retrieving the necessary scan-specific and patient-specific data from the worklist database 37 and configuring the acquisition software 31 with that data. This procedure can be commanded by the operator in a simple manner, e.g., with one click of a mouse or other device, so that the operator merely clicks a single button to determine the next patient and configure the scanner for that patient.

After the acquisition software 31 has been configured, the work station allows the operator to verify the identity of the patient arriving at the scanner and to confirm that the arriving patient is the same patient identified as the next patient by patient schedule software 33. If desired, the software can include a functionality to require the operator to confirm the identity of the arriving patient, e.g., with a single action such as a mouse click. The operator can then begin the scan, again by clicking one button, for example. Thus, as soon as the first scan ends, the operator can begin the second scan by: (1) directing the first patient to leave, (2) inviting the second patient to get on the scanner, (3) clicking the next patient button 66 to identify the next patient, retrieve all the relevant scan information, and configure the acquisition software 31 for the scan, (4) asking the second patient his or her name, (5) clicking a confirmation button to indicate that the patent in the scanner is the correct patient, and (6) clicking a button to start the scan.

In addition to providing the opportunity for increased efficiency of use, exemplary embodiments of the invention can provide other advantages. For example, the probability of operator errors in data entry may be reduced, because the operator does not experience the pressure of entering the data quickly while the patient is waiting for the scan to begin. Instead, the operator typically has ample time during the data acquisition phase of a previous scan to enter all the data for a subsequent scan. Furthermore, the probability of errors may be reduced because much of the data for configuring the scan is electronically received from the CIS 40 rather than being manually entered by the operator. Finally, the efficiency may be improved by the scheduling preferences, because the operator does not have to look through the CIS list of scheduled appointments and manually determine which patient should be scanned next based on the scheduled exam time and other factors such as a tracer injection time. Rather, the next patient is determined automatically by the patient schedule software 33 based on the preferences stored by the operator in the preferences database 39.

While the foregoing specification illustrates and describes the preferred embodiments of this invention, it is to be understood that the invention is not limited to the precise construction disclosed herein. The invention can be embodied in other specific forms without departing from the spirit or attributes. Accordingly, reference should be made to the following claims, rather than to the foregoing specification, as indicating the scope of the invention.

1. A method of configuring a scan in an imaging device, the method comprising the steps of:
beginning a data acquisition step for a first scan;
during the data acquisition step for the first scan, completing a data entry step relating to a second scan;
completing the data acquisition step for the first scan; and
beginning a data acquisition step for the second scan.

2. The method of claim 1, wherein the data entry step comprises entering all data necessary for the imaging device to begin the second scan.

3. The method of claim 1, wherein the step of beginning the data acquisition step for the second scan comprises:
commanding the imaging device to determine a next patient to be scanned;
verifying the identity of the patient arriving at the scanner; and
commanding the imaging device to begin the second scan.

4. The method of claim 3, further comprising the step of specifying at least one criterion for determining a next patient to be scanned.

5. The method of claim 1, wherein the data entry step comprises:
downloading information from a central database; and
entering data locally at a site where the scan takes place.

6. The method of claim 5, wherein the step of entering data locally comprises entering radioactive tracer information.

7. The method of claim 5, wherein the step of entering data locally comprises entering scan protocol data.

8. An imaging system comprising:
a detector which detects radiation during a data acquisition step of a scan;
at least one processor which controls configuration and execution of the scan; and
at least one memory which stores at least one computer program for executing the scan and data for configuration of the scan;
wherein the processor is programmed to conduct the data acquisition step for a first scan, and during the data acquisition step for the first scan, conduct a data entry step for a second scan.

9. The imaging system of claim 8, wherein the system comprises a medical imaging device.

10. The imaging system of claim 8, wherein the system comprises a positron emission tomography scanner.

11. The imaging system of claim 8, wherein the system comprises a single photon emission computed tomography scanner.

12. The imaging system of claim 8, wherein the system comprises an X-ray imager.

13. The imaging system of claim 8, wherein the system comprises a computed tomography scanner.

14. The imaging system of claim 8, wherein the system comprises a magnetic resonance imaging scanner.

15. The imaging system of claim 8, wherein the at least one processor is programmed to allow an operator to specify at least one criterion for determining a next patient to be scanned.

16. The imaging system of claim 8, wherein the processor is programmed to:
download information from a central database; and
receive data entered at a site where the scan takes place.

17. The system of claim 16, wherein the data entered at the site where the scan takes place comprises radioactive tracer information.

18. The system of claim 16, wherein the data entered at the site where the scan takes place comprises scan protocol data.

19. A method for configuring an imaging device comprising the steps of:
specifying at least one criterion for determining a next patient to be scanned from a plurality of scheduled patients;
querying a database with the at least one criterion; and
receiving an identification of the next patient to be scanned based on the at least one criterion.

20. The method of claim 19, wherein the at least one criterion comprises a tracer injection time.

21. The method of claim 19, wherein the at least one criterion comprises a patient arrival time.

22. The method of claim 19, wherein the at least one criterion comprises a patient registration time.

23. The method of claim 19, wherein the at least one criterion comprises a scheduled exam time.

24. The method of claim 19, wherein the at least one criterion determines a scanning order for a plurality of scheduled patients, and the method further comprises the step of receiving a scanning order for the plurality of scheduled patients based on the at least one criterion.

25. The method of claim 19, further comprising the steps of:
conducting a data acquisition step for a first scan;
during the data acquisition step for the first scan, conducting a data entry step relating to a second scan.

26. The method of claim 25, wherein the data entry step comprises:
downloading information from a central database; and
entering data locally at a site where the scan takes place.

27. The method of claim 26, wherein the step of entering data locally comprises entering radioactive tracer information.

28. The method of claim 26, wherein the step of entering data locally comprises entering data relating to a scan protocol.

29. The method of claim 25,
wherein the data entry step for the second scan is completed prior to completion of the data acquisition step of the first scan; and
wherein the step of querying the database is executed by the operator with one action; and
wherein the method further comprises the step of commanding the imaging device to begin the second scan with a single action.

30. An imaging system comprising:
a detector which detects radiation during a data acquisition step of a scan;
at least one processor which controls configuration and execution of the scan; and
at least one memory which stores at least one computer program for executing the scan and data for configuration of the scan;
wherein the processor is programmed to allow an operator to specify at least one criterion for determining a next patient to be scanned from a plurality of scheduled patients, query a patient database with the at least one criterion, and receive an identification of the next patient to be scanned based on the at least one criterion.

31. The system of claim 30, wherein the processor is programmed to generate a scanning order for the plurality of scheduled patients based on the at least one criterion.

32. The imaging system of claim 30, wherein the at least one criterion comprises a tracer injection time.

33. The imaging system of claim 30, wherein the at least one criterion comprises a patient arrival time.

34. The imaging system of claim 30, wherein the at least one criterion comprises a patient registration time.

35. The imaging system of claim 30, wherein the at least one criterion comprises a scheduled exam time.
